(19) United States
(12) Patent Application Publication
(10) Pub. No.: US 2025/0251395 A1
(43) Pub. Date: Aug. 7, 2025

(54) MOLECULES FOR DETECTION OF BIOMARKERS IN BODILY EMISSIONS

(71) Applicant: OUTSENSE DIAGNOSTICS LTD., Tel-Aviv (IL)

(72) Inventors: Yaara KAPP-BARNEA, Nirit (IL); Guy HADASSI, Tel Aviv (IL)

(21) Appl. No.: 19/041,133

(22) Filed: Jan. 30, 2025

Related U.S. Application Data
(60) Provisional application No. 63/548,651, filed on Feb. 1, 2024.

(51) Int. Cl. G01N 33/543; G01N 21/17; G01N 21/78; G01N 33/493; G01N 33/574; G01N 33/76; G06T 7/90

(52) U.S. Cl. CPC G01N 33/54386; G01N 21/78; G01N 33/493; G01N 33/57434; G01N 33/57488; G01N 33/76; G06T 7/90; G01N 2021/1765; G01N 2333/58; G01N 2333/59

(57) ABSTRACT

Apparatus and methods are described for use with a bodily emission of a subject and a toilet bowl containing water. A plurality of molecules are disposed on a substrate, each of the molecules comprising an epitope that is configured to target a biomarker and a color reporter molecule conjugated to the epitope. The color reporter molecule is configured to undergo a color change from a first color to a second color in response to being in reactive proximity with the biomarker within the water within the toilet bowl. Other applications are also described.

### CROSS-REFERENCE TO RELATED APPLICATIONS

The present application claims the benefit of U.S. Provisional Patent Application No. 63/548,651 to Kapp-Barnea, filed Feb. 1, 2024, entitled “Biosynthetic molecules for detection of biomarkers in bodily emissions,” which is incorporated herein by reference.

### FIELD OF EMBODIMENTS OF THE DISCLOSURE

Some applications of the present disclosure generally relate to analysis of bodily emissions. Specifically, some applications of the present disclosure relate to apparatus and methods for detecting biomarkers in a subject's bodily emission.

### BACKGROUND

There are substances that may be found in a human bodily emissions that are indicative of certain conditions. Some of these conditions are applicable to both male and female subjects.

For example, a subject's urine, fecal matter, vaginal fluids, saliva, nasal mucus, mucus, phlegm, sweat, and/or tears may contain one or more physical properties, chemical compounds, and/or microscopic components indicative of an underlying condition.

### SUMMARY OF EMBODIMENTS

In accordance with some applications of the present disclosure, a substrate includes one or more molecules disposed thereon. For example, the molecules include biosynthetic molecules, chemical molecules, antibodies, etc. For some applications, the substrate is flushable in the toilet bowl. For example, the substrate may include toilet paper, wipes (such as facial wipes), tampons, flushable pads, sanitary products, and/or a different type of substrate. For some applications, the molecules include reporter-conjugated epitopes that are configured to target a biomarker that may be present within a bodily emission (e.g., urine, feces, vaginal fluids, saliva, nasal mucus, mucus, phlegm, sweat, and/or tears) of the subject.

The epitopes within the molecules are conjugated to a color reporter molecule. For some applications, the color reporter molecule is configured undergo a reaction in the presence of the biomarker that produces a change of color or appearance of color. For example, the color reporter molecule is configured to undergo a conformational change, an enzymatic reaction, and/or an immunochemical reaction in the presence of the biomarker that produces a change of color or appearance of color. For some applications, the color reporter molecule is configured to form a complex with the biomarker that produces color, and/or the color reporter molecule is configured to undergo a reaction in the presence of the biomarker that forms clusters or aggregation or precipitation that produces a color change.

In some embodiments, the molecule is configured such that if the targeted biomarker is present within the bodily emission, (a) the epitope bonds to the targeted biomarker, (b) the biomarker is thereby brought into reactive proximity with the color reporter molecule, and (c) the aqueous environment of the water within the toilet bowl causes the biomarker to react with the color reporter molecule such that the color reporter molecule undergoes a color change. For some applications, the color change is detected by sensors.

For some applications, the epitopes within the molecules are conjugated to a molecule, a physical attribute of which, other than color, is configured to undergo a change upon being brought into reactive proximity with the biomarker in the aqueous environment. For example, the physical attribute may include a change in impedance, a change in the attenuation of sonic waves by the molecule, and/or a change in microwave activity of the molecule. For some applications, one or more sensors are configured to detect such changes in the physical attribute of the molecule. For some applications, a computer processor receives signals from the one or more sensors and analyzes the received signals.

For some applications, the substrate is configured to receive the bodily emission within reactive proximity of the molecules by the subject interacting with the substrate, e.g., by the subject wiping themselves (or being wiped) with the substrate. For example, when toilet paper is used as the substrate, the subject's feces and/or urine comes into reactive proximity with the molecules by the subject wiping themselves with the toilet paper. Alternatively, when facial wipes or tissues are used as the substrate, the subject's saliva, nasal mucus, mucus, phlegm, sweat, tears, and/or other emissions from the subject come into reactive proximity with the molecules by the subject wiping themselves with the substrate. Further alternatively, when toilet paper, tampons, or flushable pads are used as the substrate, the subject's vaginal fluids come into reactive proximity with the molecules by the substrate being placed into contact with, or into the vicinity of, the subject's vagina. For some applications, the substrate is placed into the toilet bowl separately from the bodily emission. For example, the subject may place the substrate in the toilet bowl before, during, and/or after urinating and/or defecating into the toilet bowl, such that the bodily emission comes into reactive proximity with the molecules upon the substrate within the toilet bowl.

As noted above, for some applications, the aqueous environment of the water within the toilet bowl causes the biomarker to react with the color reporter molecule such that the color reporter molecule undergoes a color change from a first color to a second color. Thus, although the bodily emission comes into reactive proximity with the molecules by the subject wiping themselves with the substrate, the color change occurs only once the substrate is within the toilet bowl. For some applications, the computer processor is configured to detect the presence and/or concentration of the biomarker by detecting the color change of the color reporter molecule, rather than by simply detecting the presence of the second color of the color reporter molecule. For some embodiments, by detecting the change of color rather than the presence of the second color, the computer processor is able to more accurately (e.g., with higher specificity and/or sensitivity) detect the presence and/or concentration of the biomarker within the bodily emission.

Alternatively, the computer processor is configured to detect the presence and/or concentration of the biomarker by detecting the presence of the second color of the color reporter molecule, even without detecting that the substrate has undergone a change in color.

There is therefore provided, in accordance with some embodiments of the present disclosure, an apparatus for use with a bodily emission of a subject and a toilet bowl containing water, the apparatus including:
a substrate;
a plurality of molecules disposed on the substrate, each of the molecules including an epitope that is configured to target a biomarker and a color reporter molecule conjugated to the epitope,
the color reporter molecule being configured to undergo a color change from a first color to a second color in response to being in reactive proximity with the biomarker within the water within the toilet bowl.

In some embodiments, the substrate includes toilet paper that is configured to receive feces and/or urine of the subject within reactive proximity if the molecules, in response to the subject wiping themselves with the toilet paper.

In some embodiments, the substrate is configured to receive saliva of the subject within reactive proximity of the molecules, in response to the subject wiping themselves with the substrate.

In some embodiments, the substrate includes a substrate selected from the group consisting of: toilet paper, a tampon, a sanitary product, and a flushable pad, and the substrate is configured to receive vaginal fluids of the subject within reactive proximity of the molecules in response to the subject interacting with the substrate.

In some embodiments, the epitope is configured to target iodine.

In some embodiments, the epitope is configured to target human chorionic gonadotropin (hCG).

In some embodiments, the epitope is configured to target a biomarker that is indicative of whether a female subject is pre- or post-menopausal, the biomarker being selected from the group consisting of: 6-sulfatoxymelatonin (aMT6s), Follicle-stimulating hormone (FSH), Estrone (E1), and Estradiol (E2).

In some embodiments, the epitope is configured to target a biomarker that is indicative of a female subject's menstrual cycle, the biomarker being selected from the group consisting of: pregnanediol glucuronide (PdG), and luteinizing hormone (LH).

In some embodiments, the epitope is configured to target urinary prostate-specific antigen (PSA).

In some embodiments, the epitope is configured to target one or more urinary polyamines that are indicative of a likelihood of a male subject suffering from prostate cancer and/or from benign prostatic hyperplasia (BPH).

In some embodiments, the epitope is configured to target one or more urinary polyamines that are indicative of a likelihood of a subject suffering from cancer.

In some embodiments, the epitope is configured to target one or more urinary polyamines that are indicative of a likelihood of a subject suffering from a sexually-transmitted disease.

In some embodiments, the epitope is configured to target a given pathogen within the bodily emission.

In some embodiments, the epitope is configured to target one or more inflammatory biomarkers within the bodily emission.

In some embodiments, the epitope is configured to target B-Type Natriuretic Peptide within urine of the subject.

In some embodiments, the epitope is configured to target troponin within urine of the subject.

In some embodiments, the epitope is configured to target amniotic fluid.

In some embodiments, the apparatus further includes a trap that is configured to trap the substrate within toilet bowl when the toilet bowl is flushed.

In some embodiments, the trap is configured to release the substrate after a given time period even in the absence of any further action that is taken by the subject.

In some embodiments, the apparatus further includes one or more sensors configured to be disposed in the toilet bowl and to receive light from the toilet bowl, and at least one computer processor configured to detect the presence and/or concentration of the biomarker within the bodily emission based upon the light detected by the one or more sensors.

In some embodiments, the computer processor is configured to detect the presence and/or concentration of the biomarker by detecting the color change of the color reporter molecule.

In some embodiments, the computer processor is configured to detect the presence and/or concentration of the biomarker by detecting the presence of the second color of the color reporter molecule.

In some embodiments, the computer processor is configured to detect the concentration of the biomarker based on an intensity of a light signal received by the one or more sensors at the second color of the color reporter molecule.

In some embodiments, the computer processor is configured to detect the concentration of the biomarker based on a time taken for the substrate to undergo the change in color.

There is further provided, in accordance with some embodiments of the present disclosure, a method including:
placing a bodily emission of a subject in reactive proximity with a substrate, a plurality of molecules being disposed on the substrate, each the molecules including an epitope that is configured to target a biomarker and a color reporter molecule conjugated to the epitope;
causing the biomarker, if present within the bodily emission, to react with the color reporter molecule such that the color reporter molecule undergoes a color change from a first color to a second color, by placing the substrate into water within a toilet bowl; and
driving a sensor to detect light within the toilet bowl.

In some embodiments, the substrate includes toilet paper and placing the bodily emission of the subject in reactive proximity with the substrate includes bringing feces and/or urine of the subject into reactive proximity with the molecules by the subject interacting themselves with the toilet paper.

In some embodiments, placing the bodily emission of the subject in reactive proximity with the substrate includes bringing saliva of the subject into reactive proximity with the molecules by the subject interacting with the substrate.

In some embodiments, the substrate includes a substrate selected from the group consisting of: toilet paper, a tampon, a sanitary product, and a flushable pad, and placing the bodily emission of the subject in reactive proximity with the substrate includes bringing vaginal fluids of the subject into reactive proximity with the molecules by the subject interacting with the substrate.

In some embodiments, the epitope is configured to target iodine.

In some embodiments, the epitope is configured to target human chorionic gonadotropin (hCG).

In some embodiments, the epitope is configured to target a biomarker that is indicative of whether a female subject is pre- or post-menopausal, the biomarker being selected from the group consisting of: 6-sulfatoxymelatonin (aMT6s), Follicle-stimulating hormone (FSH), Estrone (E1), and Estradiol (E2).

In some embodiments, the epitope is configured to target a biomarker that is indicative of a female subject's menstrual cycle, the biomarker being selected from the group consisting of: pregnanediol glucuronide (PdG), and luteinizing hormone (LH).

In some embodiments, the epitope is configured to target urinary prostate-specific antigen (PSA).

In some embodiments, the epitope is configured to target one or more urinary polyamines that are indicative of a likelihood of a male subject suffering from prostate cancer and/or from benign prostatic hyperplasia (BPH).

In some embodiments, the epitope is configured to target one or more urinary polyamines that are indicative of a likelihood of a subject suffering from cancer.

In some embodiments, the epitope is configured to target one or more urinary polyamines that are indicative of a likelihood of a subject suffering from a sexually-transmitted disease.

In some embodiments, the epitope is configured to target a given pathogen.

In some embodiments, the epitope is configured to target one or more inflammatory markers.

In some embodiments, the epitope is configured to target troponin within urine of the subject.

In some embodiments, the epitope is configured to target amniotic fluid.

In some embodiments, the epitope is configured to target B-Type Natriuretic Peptide within urine of the subject.

In some embodiments, the method further includes, using at least one computer processor, detecting the presence and/or concentration of the biomarker within the bodily emission based upon the light detected by the one or more sensors.

In some embodiments, detecting the presence and/or concentration of the biomarker within the bodily emission based upon the light received by the one or more sensors includes detecting the presence and/or concentration of the biomarker by detecting the color change of the color reporter molecule.

In some embodiments, detecting the presence and/or concentration of the biomarker within the bodily emission based upon the light received by the one or more sensors includes detecting the presence of the second color of the color reporter molecule.

In some embodiments, detecting the presence and/or concentration of the biomarker within the bodily emission based upon the light received by the one or more sensors includes detecting the concentration of the biomarker based on an intensity of a light signal received by the one or more sensors at the second color of the color reporter molecule.

In some embodiments, detecting the presence and/or concentration of the biomarker within the bodily emission based upon the light received by the one or more sensors includes detecting the concentration of the biomarker based on a time taken for the substrate to undergo the change in color.

There is further provided, in accordance with some embodiments of the present disclosure, an apparatus for use with a bodily emission of a subject and a toilet bowl containing water, the apparatus including:
a substrate;
a plurality of molecules disposed on the substrate, each of the molecules including an epitope that is configured to target a biomarker and a molecule conjugated to the epitope,
a detectable, physical attribute of the molecule being configured to undergo a change in response to coming into reactive proximity with the biomarker within the water within the toilet bowl.

The present disclosure will be more fully understood from the following detailed description of embodiments thereof, taken together with the drawings, in which:

### BRIEF DESCRIPTION OF THE DRAWINGS

FIG. 1 is a schematic illustration of a substrate that includes molecules that are used for detection of biomarkers within a bodily emission of a subject, in accordance with some applications of the present disclosure;

FIGS. 2A, 2B, 2C, and 2D are schematic illustrations of a trap for trapping a substrate within a toilet bowl, in accordance with some applications of the present disclosure; and

FIG. 3 is a flowchart showing steps of analysis that is performed on a subject's urine, in accordance with some applications of the present disclosure.

### DETAILED DESCRIPTION OF EMBODIMENTS

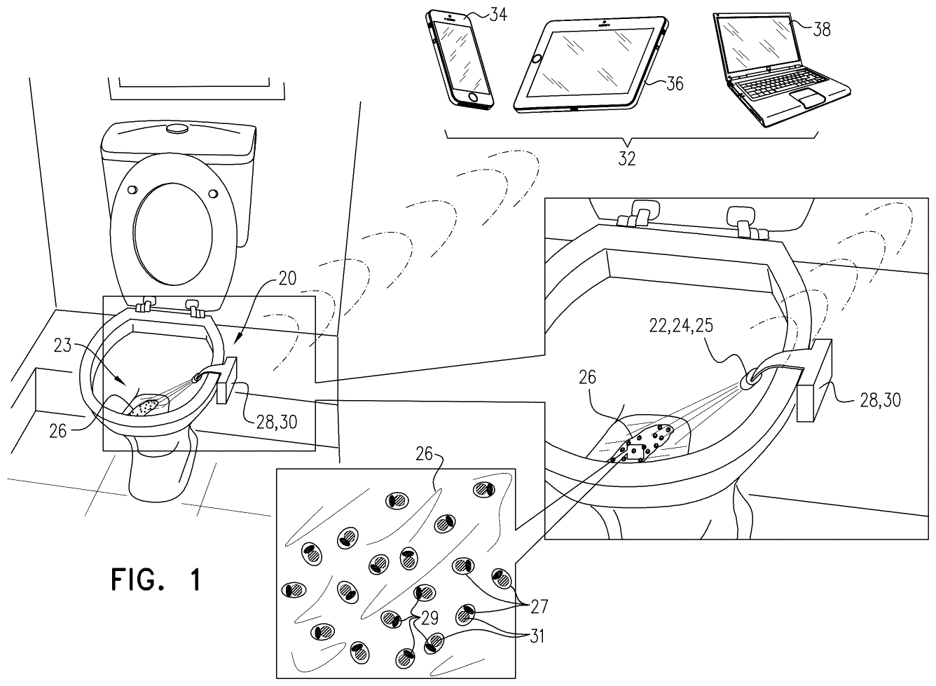

Reference is now made to FIG. 1, which is a schematic illustration of apparatus 20 for detecting biomarkers, in accordance with some applications of the present disclosure. As shown, for some applications, apparatus 20 includes a sensor module 22, which is placed inside a toilet bowl 23. For some applications (not shown), the sensor module is (and/or additional components of the apparatus are) integrated into the toilet bowl. The sensor module includes one or more sensors 24. For some applications, the sensors include an imaging component, such as an RGB camera, a spectral camera, and/or a hyperspectral camera. Alternatively or additionally, the one or more sensors may include one or more light sensors. Further alternatively or additionally, the one or more sensors may include a radar (e.g., an ultra-wideband radar), and/or an ultrasonic transducer.

For some applications, the sensors are for use with a substrate 26 that includes one or more molecules 27 disposed thereon. For example, the molecules include biosynthetic molecules, chemical molecules, antibodies, etc. For some applications, the substrate is flushable in the toilet bowl. For example, the substrate may include toilet paper, wipes (such as facial wipes), tampons, flushable pads, sanitary products, and/or a different type of substrate.

In some embodiments, molecules 27 include reporter-conjugated epitopes 29 that are configured to target a specific biomarker that may be present within a bodily emission (e.g., urine, feces, or perspiration) of the subject. The epitopes within the molecules are conjugated to a color reporter molecule 31. For some applications, the color reporter molecule is configured undergo a reaction in the presence of the biomarker that produces a change of color or appearance of color. For example, color reporter molecule 31 is configured to undergo a conformational change, an enzymatic reaction, and/or an immunochemical reaction in the presence of the biomarker that produces a change of color or appearance of color. For some applications, color reporter molecule 31 is configured to form a complex with the biomarker that produces color, and/or the color reporter molecule is configured to undergo a reaction in the presence of the biomarker that forms clusters or aggregation or precipitation that produces a color change.

In some embodiments, the molecule is configured such that if the targeted biomarker is present within the bodily emission, (a) the epitope bonds to the targeted biomarker, (b) the biomarker is thereby brought into reactive proximity with the color reporter molecule, and (c) the aqueous environment of the water within the toilet bowl causes the biomarker to react with the color reporter molecule such that the color reporter molecule undergoes a color change. For some applications, the color change is detected by sensors 24.

For some applications, the epitopes within the molecules are conjugated to a molecule, a physical attribute of which, other than color, is configured to undergo a change upon being brought into reactive proximity with the biomarker in the aqueous environment. For example, the physical attribute may include a change in impedance, a change in the attenuation of sonic waves by the molecule, and/or a change in microwave activity of the molecule. For some applications, sensors 24 include a sensor that is configured to detect such changes in the physical attribute of the molecule.

For some applications, a computer processor receives signals from the one or more sensors and analyzes the received signals. In accordance with respective applications, the computer processor that performs the analysis described herein is a computer processor 28 disposed inside housing 30 (which may also house the sensor module), or is a different computer processor that is in communication with the sensor module.

For some applications, sensor module 22 includes one or more illumination components 25. In accordance with respective applications, such illumination components include illumination components that are configured to illuminate the bodily emissions at given spectral bands (e.g., LEDs and/or lasers), and/or a broadband light source. For some applications, a broadband light source is used in combination with one or more bandpass filters (which may be used to filter the emitted and/or the detected light).

For some applications, as an alternative to or in addition to the illumination components, the sensor module includes an ultrasound transducer configured to direct ultrasonic waves into the toilet bowl, and/or a transducer (e.g., an ultrawide-beam radar transducer) configured to direct electromagnetic waves (e.g., radio waves or microwaves) into the toilet bowl.

For some applications, apparatus 20 includes a power source (e.g., battery, miniature battery, capacitor, chemical energy storage device, etc.), that is disposed outside the toilet bowl inside housing 30. Alternatively or additionally, the sensor module is connected to mains electricity (not shown). For some applications, the power source and sensor module 22 are connected wiredly, or wirelessly. In accordance with respective applications, the computer processor that performs the analysis described herein is disposed inside the toilet bowel (e.g., computer processor 28 disposed inside housing 30 (which may also house the sensor module)), or remotely. For example, as shown, the sensor module may communicate wirelessly (e.g., using a short-range wireless technology standard, such as Bluetooth®, Zigbee®, and/or other near-field communication protocols) with a user interface device 32 that includes a computer processor. Such a user interface device may include, but is not limited to, a phone 34, a tablet computer 36, a laptop computer 38, or a different sort of personal computing device. For some applications the user interface device acts as both an input device and an output device, via which the user interacts with sensor module 22. The sensor module may transmit data to the user interface device and the user interface device computer processor may run a program that is configured to analyze the received data.

For some applications, sensor module 22 and/or the user interface device 32 communicates with a remote server, e.g., to thereby communicate with a third-party device. For example, the apparatus may communicate with a physician or an insurance company over a communication network without intervention from the subject. The physician or the insurance company may evaluate the results on the third-party device and determine whether further testing or intervention is appropriate for the subject. For some applications, data relating to the received sensor signals are stored in a memory. For example, the memory may be disposed inside the toilet bowel (e.g., inside the sensor unit), inside housing 30, or remotely. Periodically, the subject may submit the stored data to a facility, such as a healthcare facility (e.g., a physician's office, or a pharmacy) or an insurance company, and a computer processor at the facility may then perform the above-described analysis on a batch of data that were acquired over a period of time.

For some applications, sensor module 22 is disposed inside a toilet bowl. For some applications, the face of the sensor module underneath which the imaging component is mounted is covered with a transparent, water-resistant cover. It is noted that FIG. 1 shows the sensor module disposed above the water level of the water within the toilet bowl. However, for some applications, at least a portion of the sensor module (e.g., the entire sensor module) is submerged within the water in the toilet bowl.

For some applications, the sensor module includes a subject sensor. The subject sensor is configured to detect when a subject is on or in the vicinity of the toilet, and/or if the subject has deposited a bodily emission and/or substrate 26 into the toilet bowl. For example, the subject sensor may include a motion sensor, configured to sense the motion of feces, urine, the subject, or the water in the toilet bowl. Alternatively or additionally, the subject sensor may include a light sensor configured to detect when the light in the bathroom is switched on, or when the subject sits on the toilet. For some applications, light sensors that are used for detecting light from the bodily emission are also used for the aforementioned function. For some such applications, the sensor module is configured to be in standby mode most of the time (such that the sensor module uses a reduced amount of power). The sensor module is switched on in response to detecting that the subject is on or in the vicinity of the toilet, and/or that the subject has defecated and/or urinated into the toilet bowl. For some applications, the imaging and/or sensing components of the sensor module acquire data in response to detecting that the subject is on or in the vicinity of the toilet, and/or that the subject has defecated and/or urinated into the toilet bowl. For some applications, the subject switches on the sensor module manually.

As described hereinabove, for some applications, sensors 24 are for use with a substrate 26 that includes one or more molecules 27 disposed thereon. For example, the molecules include biosynthetic molecules, chemical molecules, antibodies, etc. For some applications, the substrate is flushable in the toilet bowl. For example, the substrate may include toilet paper, wipes (such as facial wipes), tampons, flushable pads, sanitary products, and/or a different type of substrate. In some embodiments, molecules 27 are bonded to the substrate, for example by being lyophilized onto the substrate.

For some applications, the substrate is configured to bring the bodily emission within reactive proximity of the molecules by the subject interacting with the substrate, e.g., by the subject wiping themselves (or being wiped) with the substrate. For example, when toilet paper is used as the substrate, the subject's feces and/or urine comes into reactive proximity with the molecules by the subject wiping themselves with the toilet paper. Alternatively, when facial wipes or tissues are used as the substrate, the saliva, nasal mucus, mucus, phlegm, sweat, tears, and/or other emissions from the subject come into reactive proximity with the molecules by the subject wiping themselves with the substrate. Further alternatively, when toilet paper, tampons, or flushable pads are used as the substrate, the subject's vaginal fluids come into reactive proximity with the molecules by the substrate being placed into contact with, or into the vicinity of, the subject's vagina. For some applications, the substrate is placed into the toilet bowl separately from the bodily emission. For example, the subject may place the substrate in the toilet bowl before, during, and/or after urinating and/or defecating into the toilet bowl, such that the bodily emission comes into reactive proximity with the molecules upon the substrate within the toilet bowl.

As described hereinabove, for some applications, the epitopes within the molecules are conjugated to a molecule a physical attribute of which other than color is configured to undergo a change upon being brought into reactive proximity with the biomarker in the aqueous environment. For example, the physical attribute may include a change in impedance, attenuation of sonic waves by the molecule, and/or a change in microwave activity of the molecule. For some applications, sensors 24 include a sensor that is configured to detect such changes in the physical attribute of the molecule.

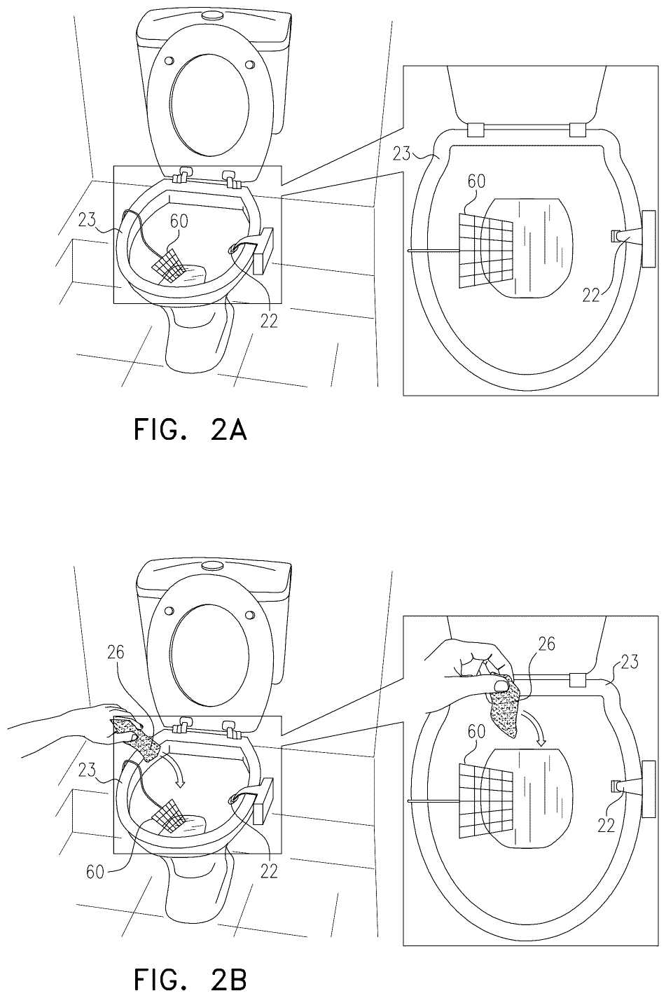

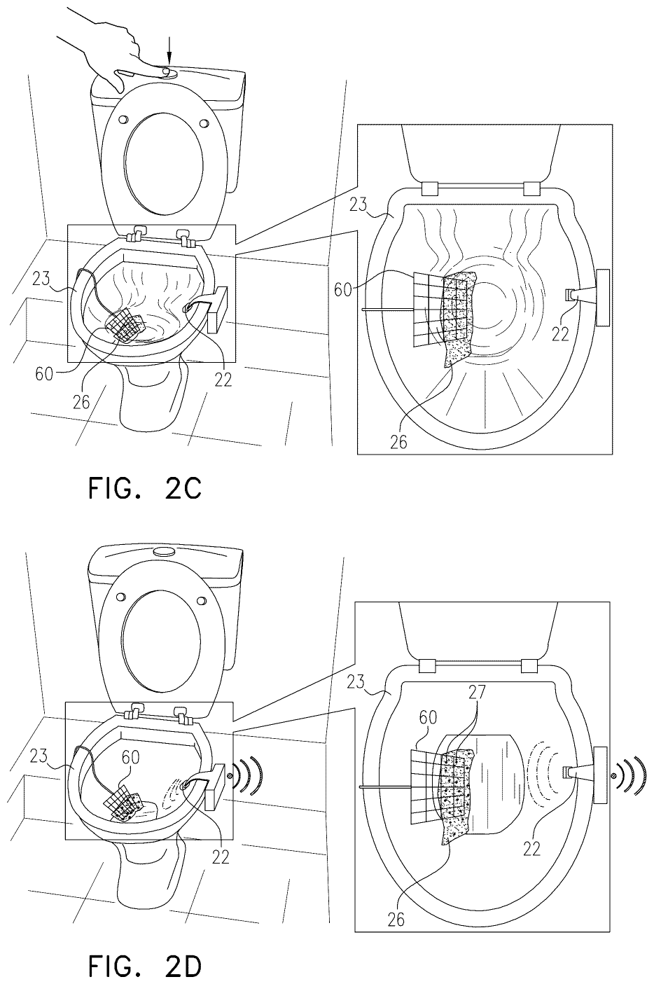

Reference is now made to FIGS. 2A-D, which are schematic illustrations of a trap 60 for trapping substrate 26 within toilet bowl 23, in accordance with some applications of the present disclosure. For such applications, assuming that the user flushes the toilet before the color reporter molecule has undergone the color change, its color change occurs in the absence of the bodily emission itself. Thus, the color change is more readily sensed by sensors 24. Therefore, in some embodiments, the trap is configured to trap the substrate within the toilet bowl for a time period after the other contents of the toilet bowl have been flushed. In some embodiments, the trap is configured to release the substrate after a given time period even in the absence of any further action that is taken by the user. For some applications, the trap is configured to release the substrate and the toilet is configured to automatically flush away the substrate after a given time period even in the absence of any further action that is taken by the user.

FIG. 2A shows trap 60 and sensor module 22 disposed within toilet bowl 23, in the absence of the substrate. FIG. 2B shows the substrate being deposited in the toilet bowl. FIG. 2C shows the toilet being flushed to remove a bodily emission (e.g., urine) from the toilet bowl. FIG. 2D shows the trap retaining the substrate in the toilet bowl even after the bodily emission has been flushed, such that the sensor module is able to detect changes in the substrate in the absence of the bodily emission, as described above.

As noted above, for some applications, the aqueous environment of the water within the toilet bowl causes the biomarker to react with the color reporter molecule such that the color reporter molecule undergoes a color change from a first color to a second color. Thus, although the bodily emission comes into reactive proximity with the molecules by the subject wiping themselves with the substrate, the color change occurs only once the substrate is within the toilet bowl. For some applications, computer processor is configured to detect the presence and/or concentration of the biomarker by detecting the color change of the color reporter molecule, rather than by simply detecting the presence of the second color of the color reporter molecule. For some embodiments, by detecting the change of color rather than the presence of the second color, the computer processor is able to more accurately (e.g., with higher specificity and/or sensitivity) detect the presence and/or concentration of the biomarker within the bodily emission. Alternatively, the computer processor is configured to detect the presence and/or concentration of the biomarker by detecting the presence of the second color of the color reporter molecule, even without detecting that the substrate has undergone a change in color.

As described hereinabove, for some applications, molecules 27 include reporter-conjugated epitopes 29 that are configured to target a specific biomarker that may be present within a bodily emission (e.g., urine or feces) of the subject. In some embodiments, in response to detecting that the substrate has undergone a change in color, the computer processor detects the presence of the biomarker within a bodily emission. For some applications, the computer processor detects the concentration of the biomarker, for example, based on an intensity of a light signal at the second color of the color reporter molecule 31, or based on the time taken for the substrate to undergo the change in color. For some applications, in response to detecting the presence of and/or a given concentration of the biomarker, the computer processor generates an output indicating a condition of the subject based upon the detected presence and/or concentration of the biomarker. Some examples of such biomarkers and the corresponding conditions are now provided.

For some applications, reporter-conjugated epitopes are configured to target iodine and the computer processor is configured to detect a presence and/or a concentration of iodine in a bodily emission (e.g., urine) of the subject by detecting a color change of the color reporter molecule within the substrate. For some such applications, the computer processor is configured to generate an output indicating whether the subject's iodine intake is insufficient, adequate, and/or excessive. For example, in response to detecting a concentration of iodine within the subject's urine that is below a threshold (with the threshold being, for example, 150 microgram/liter or less), the computer processor generates an output indicting that the subject's iodine intake is insufficient; in response to detecting a concentration of iodine within the subject's urine that is within a given range (with the range being, for example, 150-499 microgram/liter or a subrange thereof), the computer processor generates an output indicting that the subject's iodine intake is adequate; and/or in response to detecting a concentration of iodine within the subject's urine that is above a threshold (with the threshold being, for example, 500 microgram/liter or more), the computer processor generate an output indicting that the subject's iodine intake is excessive.

For some applications, reporter-conjugated epitopes are configured to target human chorionic gonadotropin (hCG) and the computer processor is configured to detect a presence and/or a concentration of human chorionic gonadotropin (hCG) in a bodily emission (e.g., urine) of the subject by detecting a color change of the color reporter molecule within the substrate. For some such applications, the computer processor is configured to generate an output indicating a likelihood of a female subject being pregnant based upon the detected concentration of human chorionic gonadotropin (hCG). For example, in response to detecting a concentration of human chorionic gonadotropin (hCG) within the subject's urine that is below a threshold (with the threshold being, for example, 70 picomole/liter or less), the computer processor generates an output indicting that the subject's iodine intake is insufficient; in response to detecting a concentration of human chorionic gonadotropin (hCG) within the subject's urine that is within a given range (with the range being, for example, 70-174 picomole/liter or a subrange thereof), the computer processor generates an output indicting that the subject's pregnancy status is unclear; and/or in response to detecting a concentration of human chorionic gonadotropin (hCG) within the subject's urine that is above a threshold (with the threshold being, for example, 174 picomole/liter or more), the computer processor generate an output indicting that the subject is likely to be pregnant.

For some applications, reporter-conjugated epitopes are configured to target 6-sulfatoxymelatonin (aMT6s), Follicle-stimulating hormone (FSH), Estrone (E1), and/or Estradiol (E2) and the computer processor is configured to detect a concentration of 6-sulfatoxymelatonin (aMT6s), Follicle-stimulating hormone (FSH), Estrone (E1), and/or Estradiol (E2) in a bodily emission of the subject by detecting a color change of the color reporter molecule within the substrate. For some applications, the computer processor is configured to determine a presence and/or a concentration of one or more of the aforementioned entities within a female subject's urine (e.g., first morning urine). For some applications, the computer processor is configured to determine a mean concentration of one or more of the aforementioned entities within the subject's urine over a given time period (e.g., a period of between one week and two months). For some such applications, the computer processor is configured to generate an output indicating whether a female subject is pre- or post-menopause based upon the concentration or mean concentration of one or more of the aforementioned entities within the subject's urine. For example, in response to detecting a concentration of 6-sulfatoxymelatonin (aMT6s), Estrone (E1), and/or Estradiol (E2) within the subject's urine that is below a threshold or that the concentration of follicle-stimulating hormone (FSH) is above a threshold, the computer processor generates an output indicting that the subject is post-menopause; and in response to detecting a concentration of 6-sulfatoxymelatonin (aMT6s), Estrone (E1), and/or Estradiol (E2) within the subject's urine that is above a threshold or that the concentration of follicle-stimulating hormone (FSH) is below a threshold, the computer processor generates an output indicting that the subject is pre-menopause.

For some applications, reporter-conjugated epitopes are configured to target pregnanediol glucuronide (PdG) and/or luteinizing hormone (LH) and the computer processor is configured to detect a presence and/or a concentration of pregnanediol glucuronide (PdG) and/or luteinizing hormone (LH) in a bodily emission of the subject by detecting a color change of the color reporter molecule within the substrate. For some applications, the computer processor is configured to determine a concentration of one or more of the aforementioned entities within a female subject's urine (e.g., first morning urine). For some such applications, the computer processor is configured to generate an output indicating a current stage of a female subject's menstrual cycle based upon the concentration of one or more of the aforementioned entities within the subject's urine. For example, in response to detecting a concentration of pregnanediol glucuronide (PdG) within the subject's urine that is above a threshold, the computer processor generates an output indicting that the subject is at the luteal stage of their menstrual cycle; and in response to detecting a concentration of pregnanediol glucuronide (PdG) within the subject's urine that is below a threshold, the computer processor generates an output indicting that the subject at the follicular stage of their menstrual cycle. Alternatively or additionally, in response to detecting a concentration of luteinizing hormone (LH) within the subject's urine that is below a threshold, the computer processor generates an output indicting that the subject is at the luteal stage of their menstrual cycle; and in response to detecting a concentration of luteinizing hormone (LH) within the subject's urine that is above a threshold, the computer processor generates an output indicting that the subject at the follicular stage of their menstrual cycle.

For some applications, reporter-conjugated epitopes are configured to target urinary prostate-specific antigen (PSA) and the computer processor is configured to detect a presence and/or a concentration of urinary prostate-specific antigen (PSA) in urine of the subject by detecting a color change of the color reporter molecule within the substrate. For some applications, the computer processor is configured to determine a concentration of urinary prostate-specific antigen (PSA) within a male subject's urine (e.g., first morning urine). For some such applications, the computer processor is configured to generate an output indicating that the subject is likely to be suffering from prostate cancer and/or from benign prostatic hyperplasia (BPH) based upon the concentration of prostate-specific antigen (PSA) within the subject's urine. For some applications, the computer processor generates an output indicating that the subject should have their serum prostate-specific antigen (PSA) measured, so that the subject can be diagnosed based upon the ratio between their urinary prostate-specific antigen (PSA) and their serum prostate-specific antigen (PSA).

For some applications, reporter-conjugated epitopes are configured to target one or more urinary polyamines (such as spermine) and the computer processor is configured to detect a concentration of the one or more urinary polyamines (such as spermine) in urine of the subject by detecting a color change of the color reporter molecule within the substrate. For some applications, the computer processor is configured to determine a presence and/or a concentration of the one or more urinary polyamines (such as spermine) within a male subject's urine. For some applications, the computer processor is configured to determine a mean concentration of one or more of the aforementioned entities within the subject's urine over a given time period (e.g., a period of between 12 hours and 72 hours). For some such applications, the computer processor is configured to generate an output indicating that the subject is likely to be suffering from prostate cancer and/or from benign prostatic hyperplasia (BPH) based upon the concentration and/or mean concentration of the one or more urinary polyamines (such as spermine) within the subject's urine.

For some applications, the reporter-conjugated epitopes are configured to target one or more biomarkers that are indicative of a cancer (e.g., bladder cancer), and the computer processor is configured to detect a presence and/or a concentration of the biomarkers by detecting a color change of the color reporter molecule within the substrate. For some such applications, the computer processor is configured to generate an output indicating that the subject is likely to be suffering from the cancer based upon the concentration and/or mean concentration of the biomarker within a bodily emission of the subject (such as urine).

For some applications, the reporter-conjugated epitopes are configured to target one or more biomarkers that are indicative of a sexually-transmitted disease and/or any microbial, parasitic and/or viral infections of the gastrointestinal tract and/or the urinary tract, and the computer processor is configured to detect a presence and/or a concentration of the biomarkers by detecting a color change of the color reporter molecule within the substrate. For example, the reporter-conjugated epitopes are configured to target Neisseria gonorrhoeae. Alternatively or additionally, the reporter-conjugated epitopes are configured to target pallidum is order to detect syphilis. Further alternatively or additionally, the reporter-conjugated epitopes are configured to target 3-hydroxy-2,4,4-trimethylpentyl 2-methylpropanoate, to detect Trichomonas vaginalis. For some such applications, the computer processor is configured to generate an output indicating that the subject is likely to be suffering from the sexually-transmitted disease, a gastrointestinal infection, and/or a urinary-tract infection, based upon the concentration and/or mean concentration of the biomarker within a bodily emission of the subject (such as urine or feces).

For some applications, the reporter-conjugated epitopes are configured to target one or more biomarkers in urine such as protein (e.g., albumin) and/or creatinine. For some applications, the reporter-conjugated epitopes are configured to target one or more inflammatory biomarkers in stool (e.g., calprotectin) and/or urine (e.g., nitrites).

For some applications, the reporter-conjugated epitopes are configured to target a given type of pathogen within stool and/or urine, to identify a source of an infection. For example, the reporter-conjugated epitopes are configured to target one or more of pyuria, leukocyte esterase (LE), and/or nitrite within urine, to detect a urinary infection. Alternatively or additionally, the reporter-conjugated epitopes are configured to target calprotectin and/or lactoferrin within feces, to detect a gastrointestinal infection.

For some applications, the reporter-conjugated epitopes are configured to target B-Type Natriuretic Peptide within urine. For some such applications, in response to detecting B-Type Natriuretic Peptide within urine and/or in response to detecting that the concentration of B-Type Natriuretic Peptide within urine exceeds a threshold, the computer processor determines (and, optionally, generates an output indicating) a likelihood that the subject is suffering from congestive heart failure. For some applications, the computer processor generates an output indicating that the subject should be tested for congestive heart failure.

For some applications, the reporter-conjugated epitopes are configured to target troponin within urine. For some such applications, in response to detecting troponin within urine and/or in response to detecting that the concentration of troponin within urine exceeds a threshold, the computer processor determines (and, optionally, generates an output indicating) a likelihood that the subject is suffering from cardiac ischemia. For some applications, the computer processor generates an output indicating that the subject should be tested for cardiac ischemia.

For some applications, the reporter-conjugated epitopes are configured to target amniotic fluid. For some such applications, in response to detecting amniotic fluid on toilet paper, facial wipes, tampons, flushable pads, sanitary products, and/or tissues, the computer processor determines (and, optionally, generates an output indicating) a likelihood that the subject is suffering from Premature Rupture of Membranes (PROM) or Preterm Premature Rupture of Membranes (PPROM). For some applications, the computer processor generates an output indicating that the subject should be tested for Premature Rupture of Membranes (PROM) or Preterm Premature Rupture of Membranes (PPROM).

For some applications, the reporter-conjugated epitopes are configured to target one or more biomarkers within the subject's saliva, nasal mucus, mucus, phlegm, sweat, tears, vaginal emissions, and/or other emissions from the subject. For some applications, the substrate includes toilet paper, facial wipes, tampons, flushable pads, sanitary products, and/or tissues and one or more of the aforementioned emissions are brought into reactive proximity with the molecules by the subject interacting with the substrate, e.g., by wiping themselves with the substrate.

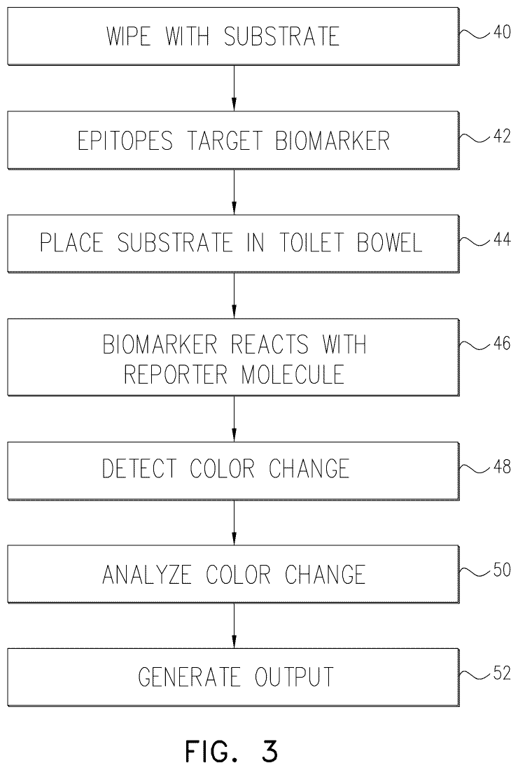

Reference is now made to FIG. 3, which is a flowchart showing steps of a method that is performed, in accordance with some applications of the present disclosure. For some applications, in a first step 40, the subject wipes a portion of their body with substrate 26, such that a bodily emission is deposited onto the substrate. In some embodiments, this causes reporter-conjugated epitopes upon the substrate to target a given biomarker within the bodily emission, in step 42. For some applications, the substrate is then placed into the toilet bowl, in step 44. In some embodiments, placement of the substrate into the aqueous environment of the water within the toilet bowl causes the biomarker to react with the color reporter molecule such that the color reporter molecule undergoes a color change, in step 46. Subsequently (in step 48), the sensors detect the color change, and (in step 50) the computer processor analyzes the detected color change and (in step 52) generates an output in response thereto.

As noted above, for some applications, the epitopes within the molecules are conjugated to a molecule a physical attribute of which other than color is configured to undergo a change upon being brought into reactive proximity with the biomarker in the aqueous environment. For example, the physical attribute may include a change in impedance, attenuation of sonic waves by the molecule, and/or a change in microwave activity of the molecule. For some applications, sensors 24 include a sensor that is configured to detect such changes in the physical attribute of the molecule.

As noted hereinabove, for some applications, the bodily emission is deposited into the toilet bowl separately from the substrate. For example, the subject may place the substrate in the toilet bowl before, during, and/or after urinating and/or defecating into the toilet bowl, such that the bodily emission comes into reactive proximity with the molecules upon the substrate within the toilet bowl. For applications, the subject does wipe themselves with the substrate, but the reporter-conjugated epitopes upon the substrate are configured to target the biomarker within the bodily emission (i.e., step 42) only once within the aqueous environment of the water within the toilet bowl. For some applications, as an alternative to or in addition to the molecules being disposed upon the substrate, the molecules are deposited into the toilet bowl via a different method. For example, cartridges containing the molecules may be disposed within the toilet (e.g., within the bowl itself or within the tank of the toilet) and the cartridges are configured to inject the molecules into the toilet bowl.

Applications of the disclosure described herein can take the form of a computer program product accessible from a computer-usable or computer-readable medium (e.g., a non-transitory computer-readable medium) providing program code for use by or in connection with a computer or any instruction execution system, such as a computer processor of user interface device 32, computer processor 28 disposed within housing 30, or a remote cloud-based computer processor. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. For some applications, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage is used.

A data processing system suitable for storing and/or executing program code will include at least one processor (e.g., a computer processor of user interface device 32, computer processor 28 disposed within housing 30, or a remote cloud-based computer processor) coupled directly or indirectly to memory elements (e.g., a memory of user interface device 32) through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the disclosure.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present disclosure may be written in any combination of one or more programming languages, including an object-oriented programming language such as Java, Smalltalk, C++ or the like and conventional procedural programming languages, such as the C programming language or similar programming languages.

It will be understood that the algorithms described herein can be implemented by computer program instructions. These computer program instructions may be provided to a processor of a general purpose computer, special purpose computer, or other programmable data processing apparatus to produce a machine, such that the instructions, which execute via the processor of the computer (e.g., a computer processor of user interface device 32, computer processor 28 disposed within housing 30, or a remote cloud-based computer processor) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

For some applications, the computer processors described herein are hardware devices programmed with computer program instructions to produce a special purpose computer. For example, when programmed to perform the algorithms described herein, the computer processor acts as a special purpose bodily-emission-analysis computer processor. For some applications, the operations described herein that are performed by computer processors transform the physical state of a memory, which is a real physical article, to have a different magnetic polarity, electrical charge, or the like depending on the technology of the memory that is used.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

1. An apparatus for use with a bodily emission of a subject and a toilet bowl containing water, the apparatus comprising:
a substrate;
a plurality of molecules disposed on the substrate, each of the molecules comprising an epitope that is configured to target a biomarker and a color reporter molecule conjugated to the epitope,
the color reporter molecule being configured to undergo a color change from a first color to a second color in response to being in reactive proximity with the biomarker within the water within the toilet bowl.

2. The apparatus according to claim 1, wherein the substrate comprises toilet paper that is configured to receive feces and/or urine of the subject within reactive proximity if the molecules, in response to the subject wiping themselves with the toilet paper.

3. The apparatus according to claim 1, wherein the substrate is configured to receive saliva of the subject within reactive proximity of the molecules, in response to the subject wiping themselves with the substrate.

4. The apparatus according to claim 1, wherein the substrate comprises a substrate selected from the group consisting of: toilet paper, a tampon, a sanitary product, and a flushable pad, and wherein the substrate is configured to receive vaginal fluids of the subject within reactive proximity of the molecules in response to the subject interacting with the substrate.

5. The apparatus according to claim 1, wherein the epitope is configured to target iodine.

6. The apparatus according to claim 1, wherein the epitope is configured to target human chorionic gonadotropin (hCG).

7. The apparatus according to claim 1, wherein the epitope is configured to target a biomarker that is indicative of whether a female subject is pre- or post-menopausal, the biomarker being selected from the group consisting of: 6-sulfatoxymelatonin (aMT6s), Follicle-stimulating hormone (FSH), Estrone (E1), and Estradiol (E2).

8. The apparatus according to claim 1, wherein the epitope is configured to target a biomarker that is indicative of a female subject's menstrual cycle, the biomarker being selected from the group consisting of: pregnanediol glucuronide (PdG), and luteinizing hormone (LH).

9. The apparatus according to claim 1, wherein the epitope is configured to target one or more targets selected from the group consisting of:
urinary prostate-specific antigen (PSA),
one or more urinary polyamines that are indicative of a likelihood of a male subject suffering from prostate cancer and/or from benign prostatic hyperplasia (BPH),
one or more urinary polyamines that are indicative of a likelihood of a subject suffering from cancer,
one or more urinary polyamines that are indicative of a likelihood of a subject suffering from a sexually-transmitted disease,
a given pathogen within the bodily emission, and
one or more inflammatory biomarkers within the bodily emission.

10-14. (canceled)

15. The apparatus according to claim 1, wherein the epitope is configured to target one or more markers within urine of a subject, selected from the group consisting of: B-Type Natriuretic Peptide, and troponin.

16. (canceled)

17. The apparatus according to claim 1, wherein the epitope is configured to target amniotic fluid.

18. The apparatus according to claim 1, further comprising a trap that is configured to trap the substrate within toilet bowl when the toilet bowl is flushed.

19. The apparatus according to claim 18, wherein the trap is configured to release the substrate after a given time period even in the absence of any further action that is taken by the subject.

20. The apparatus according to claim 1, further comprising one or more sensors configured to be disposed in the toilet bowl and to receive light from the toilet bowl, and at least one computer processor configured to detect the presence and/or concentration of the biomarker within the bodily emission based upon the light detected by the one or more sensors.

21. The apparatus according to claim 20, wherein the computer processor is configured to detect the presence and/or concentration of the biomarker by detecting the color change of the color reporter molecule.

22. The apparatus according to claim 20, wherein the computer processor is configured to detect the presence and/or concentration of the biomarker by detecting the presence of the second color of the color reporter molecule.

23. The apparatus according to claim 20, wherein the computer processor is configured to detect the concentration of the biomarker based on an intensity of a light signal received by the one or more sensors at the second color of the color reporter molecule.

24. The apparatus according to claim 20, wherein the computer processor is configured to detect the concentration of the biomarker based on a time taken for the substrate to undergo the change in color.

25. A method comprising:
placing a bodily emission of a subject in reactive proximity with a substrate, a plurality of molecules being disposed on the substrate, each the molecules comprising an epitope that is configured to target a biomarker and a color reporter molecule conjugated to the epitope;
causing the biomarker, if present within the bodily emission, to react with the color reporter molecule such that the color reporter molecule undergoes a color change from a first color to a second color, by placing the substrate into water within a toilet bowl; and
driving a sensor to detect light within the toilet bowl.

26-46. (canceled)

47. An apparatus for use with a bodily emission of a subject and a toilet bowl containing water, the apparatus comprising:
a substrate;
a plurality of molecules disposed on the substrate, each of the molecules comprising an epitope that is configured to target a biomarker and a molecule conjugated to the epitope,
a detectable, physical attribute of the molecule being configured to undergo a change in response to coming into reactive proximity with the biomarker within the water within the toilet bowl.
